(19) United States
(12) Patent Application Publication
(10) Pub. No.: US 2011/0152177 A1
(43) Pub. Date: Jun. 23, 2011

(54) Functional Feed Composition

(76) Inventors: Jose Luis Vecino, Stavanger (NO); Simon Wadsworth, Hommersak (NO)

(21) Appl. No.: 12/988,792

(22) PCT Filed: Apr. 24, 2009
(86) PCT No.: PCT/NO2009/000156

§ 371 Date: Mar. 13, 2011

(30) Foreign Application Priority Data
Apr. 24, 2008 (NO) 2008 1977

(51) Int. Cl. A61K 38/14; A61P 31/00; A61P 29/00

(52) U.S. Cl. 514/3.1; 514/20.9; 514/2.3; 514/3.7; 514/4.4

(57) ABSTRACT

A feed composition comprising conventional feed ingredients, peptidoglycan and nucleotides is described. The feed composition can be used to prevent or reduce infectious diseases, and prevent symptoms related to an infectious disease in an animal. There is also provided a method for feeding of fish by providing this feed composition in the period previous of the challenge by an infection, during the infection or after the infection.

### FIELD OF THE INVENTION

The present invention relates to a feed composition, which can be administered to prevent or reduce the symptoms of infectious diseases in animals, the use of the feed composition and a method for feeding of fish as defined in the preamble of the independent claims.

### BACKGROUND OF THE INVENTION

Piscirickettsia salmonis is a small, intracellular bacterium that causes a fatal septicaemia in salmonids. Since the initial isolation in the late 1980's, P. salmonis has been the primary cause of mortality of salmonids in the aquaculture industry in Chile. Because P. salmonis is intracellular, efficiency of antibiotic treatment of infected salmon is poor. Vaccine development has also proved difficult due to the intracellular nature of the bacterium. Current vaccines against P. salmonis are therefore largely ineffective after 6 months after the transfer from freshwater to seawater and the industry is still depending to a large degree on antibiotics, when treating this disease. Use of antimicrobial agents, such as oxolinic acid, has been extensive for a number of years in Chile, reaching over 130 t (active compound) per year. Over 80% of this volume is used to control P. salmonis, although no antimicrobial has proven to be consistently effective.

Moreover, antibiotic treatments such as with oxytetracycline and oxolinic acid can significantly reduce the functions of the specific and non-specific immune system of fish such as the Atlantic salmon. This immune suppression increases the risk of re-infection by pathological organisms such as by P. salmonis, leading to a rapid re-colonization of P. salmonis and repeated requirement of antibiotic administration. This extensive re-use of antibiotics further reduces the immunity of the fish and affects the natural microbial flora of the organisms such as the gut microflora. Lower feeding efficiency and growth are some of the reported negative effects of antibiotic treatments in this context and are thus not only an economical issue but also an issue of general fish welfare. Increasing public and consumer awareness of fish welfare increases the need for effective alternative treatments and prevention of disease outbreaks in aquaculture production.

The continued use of antibiotics has also raised residue and food safety concerns. It is now a requirement for all batches of harvested salmon from Chile for both the US and Japanese markets to be tested for antibiotic residues. These initiatives have resulted in a longer withdrawal period of over 2 months for Atlantic salmon populations prior to harvest. This period therefore represents a significant risk and can lead to high losses of valuable fish in case of serious infections of all kind. Moreover, any infection and losses in this pre-harvest population can result in significant financial penalty as no antibiotics can be used to control the disease.

Immune stimulants enhancing the immune response of fish have an important application in aquaculture, especially when deployed as part of an integrated health management program. Peptidoglycans (PGs) are structural components of bacterial cell walls and can form up to 90% dry weight of gram-positive bacteria. PGs are responsible for cell strength, shape and counteracting the osmotic pressure of the cytoplasm. They are formed from two alternating amino sugars that produce a strong lattice type structure. Due to their presence in many pathogenic bacteria PGs produce a strong response when exposed to a host's immune system. PGs have demonstrated improved protection against pathogens in a range of juvenile as well as adult aquaculture species including salmonids, yellowtail, tilapia, flounder and shrimp. However, it has been shown that the administration of peptidoglycan can result in negative side effects such as reduced survival, if it is not administered in an optimal dose, such as being too high or too low, and especially if administered for longer periods to the organism. From an aquaculture point of view, it would be beneficial if this strong immune stimulant can be administered for a longer period than what is recommendable based upon today's experimental experiences and knowledge. Matsuo K. & Miyazono I. (“The influence of long-term administration of peptidoglycan on disease resistance and growth of juvenile Rainbow-trout. Nippon Suisan Gakkaishi 89 (8): 1377-1379, August 1993) reported that oral administration of PG longer administration than 28 days can lead to decrease in disease resistance.

Most cells produce nucleotides for use in cell replication. Under normal conditions in any living organism the constant synthesis of nucleotides is well balanced with the natural demands needed for cell proliferation.

The problem to be solved by the present invention is to develop and provide an effective functional health diet and feeding regime without the above mentioned negative side effects and which improves survival of fish during periods when the fish is or has been exposed to infections such as by P. salmonis. Such a diet would result in considerable economical benefits to both fish and feed producers. Moreover, it will contribute to improve fish well fare and can reduce the need and amount of antibiotic administration with all the known disadvantages.

### SUMMARY OF THE INVENTION

According to a first aspect of the invention, there is provided a feed composition comprising conventional feed ingredients characterized in that the composition further comprises peptidoglycan and nucleotides.

Preferable, peptidoglucan in the feed composition is in the range of 0.001-0.01%, more preferably 0.001-0.005% and most preferably 0.001% by weight. Preferably, the nucleotides are in the range of 0.05%-1% by weight, more preferably 0.1-0.5% by weight and most preferably of 0.2% by weight.

Preferable, the weight ratio of nucleotides to peptidoglucan is more than 4, more preferable more than 20, and more preferable more than 40.

Preferable, the weight ratio of nucleotides to peptidoglucan is more than 40.

Preferable, the weight ratio of nucleotides to peptidoglucan is more than 20, and the amount of peptidoglucan is in the range of 0.001-0.01% of the weight of the feed composition.

Preferable, the weight ratio of nucleotides to peptidoglucan is more than 40, and the amount of peptidoglucan is in the range of 0.001-0.01% of the weight of the feed composition.

Preferable, the weight ratio of nucleotides to peptidoglucan is more than 20, and the amount of peptidoglucan is less than 0.01%, preferable about 0.05 or less, related to the weight of the feed composition.

Preferable, the weight ratio of nucleotides to peptidoglucan is more than 40, and the amount of peptidoglucan is less than 0.01%, preferable about 0.005% or less, related to the weight of the feed composition.

Preferable, the amount of nucleotides is about 0.2% and the amount of peptidoglucan is about 0.005% of the total weight of the feed composition.

Preferable, the nucleotides are chosen from adenosine monophosphate, cytidine monophosphate, guanosine monophosphate, uridine monophosphate, thymidine monophosphate.

Preferable, the composition further comprises other immune stimulating and/or anti-inflammatory ingredients.

According to a second aspect of the invention, the feed composition comprising conventional feed ingredients, nucleotides, and peptidoglycan is used as a functional feed composition to prevent or reduce the symptoms related to an infectious disease in an animal.

According to a third aspect of the invention, a feed composition comprising conventional feed ingredients and peptidoglycan is used as a functional feed composition to prevent or reduce the symptoms related of an infectious disease in an animal wherein the concentration of peptidoglycan is in the range of 0.01 g kg⁻¹ feed to 0.05 g kg⁻¹ feed.

Preferable, the feed compositions of the second and third aspect are used to increase survival and/or growth in fish challenged by an infection.

More preferable, the compositions are used in cases when the infection is caused by bacteria such as Piscirikettsia salmonis, Moritella viscose, Francisella sp, Mouth Rot, Streptococcal infections, Vibrio infections; Pancreas disease, NSAV, gill inflammation (GI), heart and skeletal muscle inflammation (HSMI), infectious salmonid anaemia (ISA) virus, Saprolegniosis, and sealice infestation.

Preferable, the compositions are used, when the animal is an aquatic animal and more preferably when the aquatic animal is a fish, crustacean or mollusk. The fish can be a salmonid, flat fish, or any other culturable fish species and most preferred the fish is Atlantic salmon Salmo salar.

It is also preferred that the compositions are used for the production of a medicament for the prophylaxis and/or treatment of infectious diseases in animals, and/or nutraceutical, and/or functional feed and/or for the reduction of symptoms of a disease.

The compositions can also be used for the effective recovery following an infection and/or antibiotic treatment due to an infection and/or to reduce the risk of a re-infection.

The compositions can also be used for the improved treatment of an infection in combination with antibiotics and/or previous of an antibiotic treatment.

In a fourth aspect of the invention, there is provided a method for feeding of fish which are susceptible to an infection characterized in that a composition according to any of the claims 1-5 is provided to the fish in the period previous of the challenge by an infection, during the infection or after the fish has been infected.

Preferable, the composition is fed for a period of 1-12 weeks prior to the infection, more preferably of 2-6 weeks, most preferably of 4 weeks.

Alternatively, the fish can be fed a conventional feed composition further comprising nucleotides for a defined period followed by feeding of the composition according to any of the claims 1-12.

Preferable, the feed composition further comprising nucleotides is fed for a period of 1-8 weeks, more preferably of 2 to 4 weeks, most preferably of 3 weeks.

It is preferred that the feed composition according to any of the claims 1-12 is fed for a period of 1-12 weeks, most preferably of 1 week.

Preferable, the methods defined in the method claims are applied when the infection is caused by bacteria such as Piscirikettsia salmonis, Moritella viscose, Francisella sp, Mouth Rot, Streptococcal infections, Vibrio infections; Pancreas disease, NSAV, gill inflammation (GI), heart and skeletal muscle inflammation (HSMI), infectious salmonid anaemia (ISA) virus, Saprolegniosis, and sealice infestation.

Preferred embodiments are also defined in the dependent claims.

### DESCRIPTION OF THE INVENTION

Embodiments of the invention will now be described, by the way of examples with reference to the following diagrams, wherein:

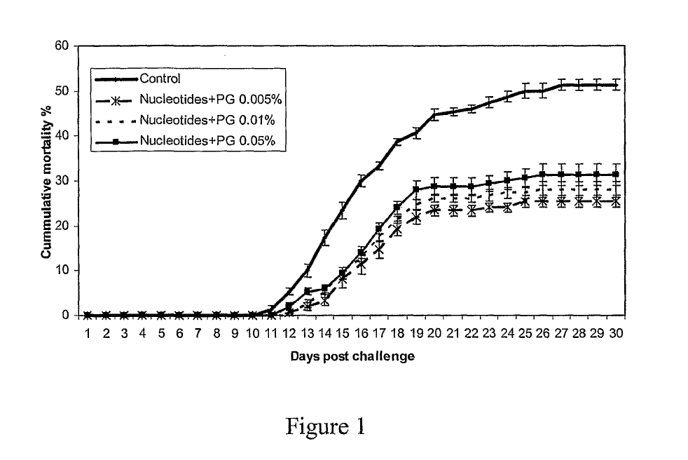

FIG. 1 shows the cumulative mortality (mean±SEM) in experiment 1 during the post-challenge period of Atlantic Salmon (Salmo salar) fed different diets in the pre-challenge phase.

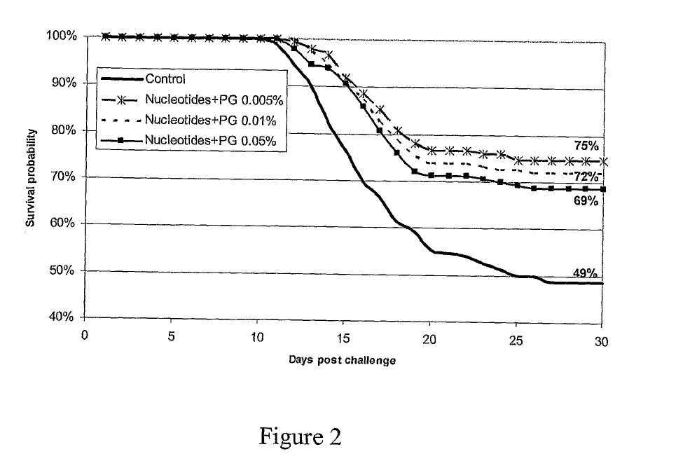

FIG. 2 shows the Kaplan-Meier survival curves (Relative Percentage of Survival, mean±standard error of the mean) of Atlantic salmon (Salmo salar) during the post-challenge period (experiment 1) after being fed different diets in the pre-challenge phase.

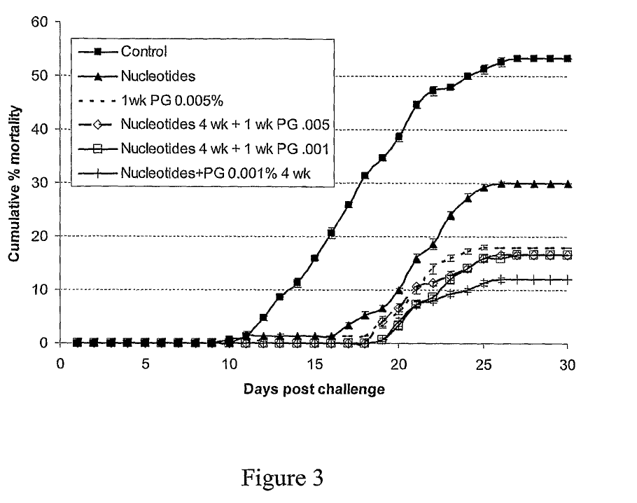

FIG. 3 shows the cumulative mortality (%) (mean±SDEV) of Atlantic salmon (Salmo salar) in experiment 2 expressed as time series. Fish were fed different diets prior to the Piscirickettsia salmonis challenge.

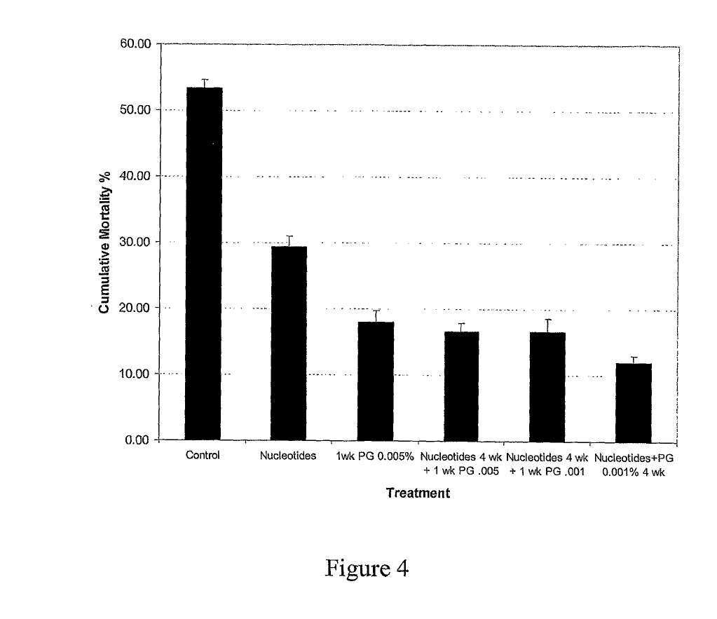

FIG. 4 shows the cumulative mortality (%) (mean±SEM) of Atlantic Salmon (Salmo salar) in experiment 2 expressed as final mortality after the post-challenge phase. Fish were fed different diets prior to the challenge with Piscirickettsia salmonis.

### EXPERIMENTAL SECTION

### Experiment 1

A total of 640 Atlantic salmon Salmo salar (160 fish per group), mixed sex, start weight 80 g, seawater adapted, were fed for a period for 4 weeks at 2% body weight per day. In practice, the fish has free access to feed. Fish were maintained in 6 tanks. The fish were pit-tagged to identify the different groups, before the start of the feeding period. Fish were maintained in ambient seawater temperature (16° C.). Mortalities were assessed on a daily basis.

There were 4 different dietary treatments in the pre-challenge period (Table 3) including 5 different diets (Table 1). Three groups of fish were fed a diet comprising nucleotides for 3 weeks followed by three different diets comprising peptidoglycan in different concentrations and nucleotides for one week. The control group received a commercial diet during the whole pre-challenge period of 4 weeks. All diets comprised conventional feed ingredients and did only differ in their amount of added peptidoglycan and nucleotides (Table 2).

Challenge with Piscirikettsia salmonis and Post-Challenge Feeding

At 24 hours after the final feeding in the pre-challenge period the fish were mixed equally into 6 tanks with 25 fish per group per tank (100 fish total per tank). Fish were challenged with 0.2 ml of a lethal dose (LD₅₀) of Piscirikettsia salmonis by intra-peritoneal injection. After the challenge fish were fed the same control diet without any added nucleotides or peptidoglycan for 30 days (post-challenge) when the experiment was terminated and the final mortalities assessed.

TABLE 1
Test diets used in experiment 1.

| Diet | |
|---|---|
| No. | Diet name |
| 1. | Control diet (Commercial) |
| 2. | Diet with 0.2% nucleotides and PG 0.005%; (50 g PG tonne⁻¹) |
| 3. | Diet with 0.2% nucleotides and PG 0.01%; (100 g PG tonne⁻¹) |
| 4. | Diet with 0.2% nucleotides and PG 0.05%; (500 g PG tonne⁻¹) |
| 5. | Diet with 0.2% nucleotides |

PG = peptidoglycan.

TABLE 2
Main feed ingredients and chemical analysis of the
composition of the test diets in experiment 1.

| | Diet 1 | Diet 2 | Diet 3 | Diet 4 | Diet 5 |
|---|---|---|---|---|---|
| Ingredient | | | | | |
| (% by weight)) | | | | | |
| Fish meal | 51 | 51 | 51 | 51 | 51 |
| Fish oil | 18 | 18 | 18 | 18 | 18 |
| Wheat gluten | 7 | 7 | 7 | 7 | 7 |
| Functional feed | 8 | 8 | 8 | 8 | 8 |
| composition | | | | | |
| Vitamin premix* | 0.2 | 0.2 | 0.2 | 0.2 | 0.2 |
| Mineral premix** | 0.3 | 0.3 | 0.3 | 0.3 | 0.3 |
| Peptidoglycan | — | 0.005 | 0.01 | 0.05 | — |
| Nucleotides | — | 0.2 | 0.2 | 0.2 | 0.2 |
| Composition (%) | | | | | |
| Protein | 47.9 | 47.9 | 47.9 | 47.9 | 47.9 |
| Lipid | 24.3 | 24.3 | 24.3 | 24.3 | 24.3 |
| Ash | 7.5 | 7.5 | 7.5 | 7.5 | 7.5 |
| Dry mass | 92.7 | 92.7 | 92.7 | 92.7 | 92.7 |

*EWOS Vitamin premix ®
**EWOS Mineral premix ®.

TABLE 3
Test diet feeding regimes in experiment 1 prior to the challenge
with Piscirikettsia salmonis (pre-challenge) and after the
challenge with P. salmonis (post-challenge).

| | Pre-challenge | | | | Post-challenge |
|---|---|---|---|---|---|
| Treatment | Week 1 | Week 2 | Week 3 | Week 4 | 30 days |
| 1 | Diet 1 | Diet 1 | Diet 1 | Diet 1 | Diet 1 |
| (control) | | | | | |
| 2 | Diet 5 | Diet 5 | Diet 5 | Diet 2 | |
| 3 | Diet 5 | Diet 5 | Diet 5 | Diet 3 | |
| 4 | Diet 5 | Diet 5 | Diet 5 | Diet 4 | |

### LD50 Assessment

The correct dose to determine the mortality level for 50% of the population (LD₅₀) was assessed prior to the main challenge. A total of 220 fish (Salmo salar) from the same stock of fish used in the feeding experiment were distributed in 4×700 L tanks. The fish had an average weight of 120 to 150 g (55 fish per tank). The tanks were supplied with sea water at room temperature. Once the fish were adjusted to the tank conditions they were injected (0.2 ml fish, intra-peritoneal injection, medial ventral) with 4 dilutions of a known titer of P. salmonis PSLT8 ( 1/10, 1/100, 1/1000 & 1/10,000). Water temperature and mortality data of injected fish were recorded over a 30 day-period. The estimate of the LD₅₀ was performed in parallel with the feeding period of the different diets.

### Statistics

Mortalities were expressed as cumulative mortality (%). Data was assessed using a Kaplan-Meier survival analysis, in addition to a Log Rank & Wilcoxon test (Minitab 13.32, State College, Pa., USA). Relative Percent Survival (RPS) was calculated as:

$$RPS = ( 1 - \frac{Mortality in Test group}{Mortality in Control group} ) \times 100$$

### Results and Discussion

Fish fed nucleotides and peptidoglycan (PG) had a significantly higher Relative Percent Survival (RPS) by the end of the study after 30 days post-challenge (Table 4, FIG. 2) (Log-Rank test: p<0.001; Wilcoxon test: p<0.001) compared to fish receiving the control diet. The probability of survival in the control group was below 49%, compared to 69%, 72% and 75% in the treatments receiving nucleotides and PG 0.05%, nucleotides and PG 0.01% and nucleotides and PG 0.005%, respectively (FIG. 2). There were no significant differences in mortality between the treatments receiving diets comprising a combination of nucleotides and peptidoglycan, although a clear trend for lower mortalities with lower dose was observed (FIG. 1). There was low deviation in mortalities between tanks and no significant difference of tank effect was observed (Log-Rank test: p=0.906; Wilcoxon test: p=0.952).

TABLE 4
Relative Percent Survival (RPS) and mortality percentage at
the end of the post-challenge phase (mean ± standard error
of the mean) of Atlantic salmon (Salmo salar) which were fed
different diets prior to the P. salmonis challenge.

| Treatment 1 | RPS (%) | Mortality (%) |
|---|---|---|
| 1 | — | 51 ± 1.23 |
| (control) | | |
| 2 | 51 | 25 ± 1.33* |
| 3 | 46 | 28 ± 1.79* |
| 4 | 40 | 31 ± 2.40* |

*indicates significant differences (p < 0.001 compared to the control.

### Experiment 2

A total of 900 Atlantic salmon Salmo salar (150 fish per treatment, 6 treatments), start weight 80 g, seawater adapted, were pit-tagged to identify the different groups, before the start of the feeding period. Fish were maintained in ambient seawater temperature (13° C.). Fish were fed for a period for 8 weeks at 2% body weight per day with six different diets in different combinations: a control diet without additional ingredients, a diet comprising nucleotides, a diet comprising peptidoglycan and diets comprising combinations of nucleotides and peptidoglycan (Table 5). Apart from the addition of nucleotides and/or peptidolgycan, all diets contained conventional feed ingredients (Table 6). In practice, the fish has free access to feed.

TABLE 5
Test diets used in experiment 2.

| Diet | |
|---|---|
| No. | Diet name |
| 1. | Control diet (Commercial) |
| 2. | Diet with 0.2% nucleotides |
| 3. | Diet with PG 0.005%; (50 g PG tonne⁻¹) |
| 4. | Diet with 0.2% nucleotides + PG 0.005% (50 g PG tonne⁻¹) |
| 5. | Diet with 0.2% nucleotides + PG 0.001% (10 g PG tonne⁻¹) |

PG = peptidoglycan.

TABLE 6
Main feed ingredients and chemical analysis of the
composition of the test diets in experiment 2.

| | Diet 1 | Diet 2 | Diet 3 | Diet 4 | Diet 5 |
|---|---|---|---|---|---|
| Ingredient | | | | | |
| (% by weight) | | | | | |
| Fish meal | 51 | 51 | 51 | 51 | 51 |
| Fish oil | 18 | 18 | 18 | 18 | 18 |
| Wheat gluten | 7 | 7 | 7 | 7 | 7 |
| Corn gluten meal | 8 | 8 | 8 | 8 | 8 |
| Vitamin premix* | 0.2 | 0.2 | 0.2 | 0.2 | 0.2 |
| Mineral premix** | 0.3 | 0.3 | 0.3 | 0.3 | 0.3 |
| Peptidoglycan | — | — | 0.005 | 0.005 | 0.001 |
| Nucleotide | — | 0.2 | — | 0.2 | 0.2 |
| Composition (%) | | | | | |
| Protein | 47.6 | 47.6 | 47.6 | 47.6 | 47.6 |
| Lipid | 24.3 | 24.3 | 24.3 | 24.3 | 24.3 |
| Ash | 7.5 | 7.5 | 7.5 | 7.5 | 7.5 |
| Dry mass | 92.7 | 92.5 | 92.7 | 92.5 | 92.7 |

*EWOS Vitamin premix ®
**EWOS Mineral premix ®.

Fish were fed the differed diets during the pre-challenge phase of 4 weeks (Table 7): Treatment 1 received a control diet without any additional ingredients during the 4 weeks of the pre-challenge period. Treatment 2 received a diet with added nucleotides for 4 weeks. In treatment 3 fish were fed the control diet for 3 weeks followed by a diet comprising 0.005% PG for 1 week. In treatment 4 and 5 fish were fed the diet comprising nucleotides for 3 weeks followed by diets comprising to different combinations of nucleotides and peptidoglycan (0.005% PG in treatment 4 and 0.001% PG in treatment 5) for 1 week. In treatment 6 fish were fed a diet comprising a combination of nucleotides and peptidoglycan for 4 weeks. After the pre-challenge period fish were challenged with P. salmonis as described in detail below.

During the post-challenge period, all treatments were fed the control diet for 4 weeks until the experiment was finished.

TABLE 7
Feeding regime in experiment 2 before the fish were challenged with
Piscirikettsia salmonis (pre-challenge) and after they were challenged
with P. salmonis (post-challenge). Positive and negative controls are
conventional commercial diets with added nucleotides (Diet 2) and
without added nucleotides (Diet 1).

| Treat- | Pre-challenge | | | | Post-challenge | | | |
|---|---|---|---|---|---|---|---|---|
| ment | Week 1 | Week 2 | Week 3 | Week 4 | Week 5 | Week 6 | Week 7 | Week 8 |
| 1 | Diet 1 | Diet 1 | Diet 1 | Diet 1 | Diet 1 | | | |
| 2 | Diet 2 | Diet 2 | Diet 2 | Diet 2 | | | | |
| 3 | Diet 1 | Diet 1 | Diet 1 | Diet 3 | | | | |
| 4 | Diet 2 | Diet 2 | Diet 2 | Diet 4 | | | | |
| 5 | Diet 2 | Diet 2 | Diet 2 | Diet 5 | | | | |
| 6 | Diet 5 | Diet 5 | Diet 5 | Diet 5 | | | | |

Challenge with Piscirikettsia salmonis

After four weeks feeding test diets (pre-challenge period), the fish were distributed equally into 6 tanks with 25 fish per group per tank (150 fish total per tank). Fish were challenged with 0.2 ml of a dose predicted to kill 50% of the population (LD₅₀) of Piscirikettsia salmonis by intra-peritoneal (i.p.) injection. Fish were fed control diets throughout the challenge period. Dead fish were removed daily and recorded.

### LD₅₀ Assessment:

The estimate of the LD₅₀ was performed as described for experiment 1 in parallel with the feeding period of the different diets. The average weight of fish for the LD₅₀ assessment was 80 g (55 fish per tank).

### Statistics

Statictical assessments were carried out as described in experiment 1.

### Results and Discussion

The LD₅₀—dose was administered to achieve a level of mortality in the control population of approximately 50%. The final control mortality reached 53% (Table 8, FIG. 4). There appeared to be no significant tank-based effect in the current challenge study (p=0.967).

At 14 days post-challenge mortalities had passed 10% in the control treatment (FIG. 3). In the groups fed peptidoglycan mortalities reached 10% after 21 days post challenge. Nucleotide addition alone also delayed, as well as reduced the overall mortality (FIG. 4), however not as efficient as it was the case for the combination of peptidoglycan and nucleotides. Fish receiving the diet only comprising nucleotides as an additive showed a Relative Percent Survival (RPS) of 45% (Table 8) and a significantly higher survival probability (p<0.001, Log Rank-Wilcoxon) compared to the control receiving the control diet without any additive.

All treatments receiving peptidoglycan had significantly higher survival probabilities (p>0.001, Log Rank-Wilcoxon) with RPS values ranging from 66% to 78% compared to the treatments receiving the control diet. (Table 8, FIG. 3).

TABLE 8
Percentage mortality and Relative Percent Survival (RPS)
of Atlantic salmon (Salmo salar) fed different diets prior
to the Piscirickettsia salmonis challenge.

| Treatment | Average mortality (%) | SEM | RPS |
|---|---|---|---|
| 1 | 53.33 | 1.33 | — |
| (control) | | | |
| 2 | 29.33 | 1.69 | 45 |
| 3 | 18.00 | 1.71 | 66 |
| 4 | 16.67 | 1.23 | 69 |
| 5 | 16.67 | 1.91 | 69 |
| 6 | 12.00 | 1.03 | 78 |

SEM = Standard error of means.

The peptidoglycan groups all had significantly different survival probabilities from the treatment receiving only nucleotides as an additive (p<0.05, Log Rank-Wilcoxon).

Fish fed diets comprising a combination of nucleotides and peptidoglycan (treatment 4, 5 and 6) in the pre-challenge phase had 3-12% higher survival probabilities compared to fish fed a diet only comprising peptidoglycan (treatment 3) without any addition of nucleotides.

There were no differences in survival in fish fed a diet with nucleotides for 3 weeks followed by a diet comprising nucleotides in combination with different amounts of peptidoglycan (0.001% PG or 0.005% PG). However, best post-challenge survival was surprisingly achieved when fish were fed a combination of peptidoglycan in a low concentration (0.001% PG) in combination with nucleotides for the whole pre-challenge period of 4 weeks (treatment 6).

Conclusions from Experiment 1 and 2:

Feeding a combination of nucleotides and peptidoglycan in experiment 1 significantly increased the resistance to Piscirickettsia salmonis, compared to the negative control (Commercial diet). There was no significant difference in survival between test groups (peptidoglycan+nucleotides) although there was a trend for increased survival with lower dose in experiment 1.

Zhou et al. (2006; “Effects of dietary supplementation of A3α-peptidoglycan on innate immune responses and defense activity of Japanese flounder (Paralichthys olivaceus)”, Aquaculture 251, 172-181) found 4 g kg⁻¹ diet as the optimal dose of peptidoglycan in Japanese flounder (Paralichthys olivaceus), following challenge with Vibrio anguillarium. Doses both lower and higher than 4 g kg⁻¹ are reported to increase mortalities. Similar dose ranging effects were observed with black tiger prawns (Penaeus monodon) where improved survival was achieved at a lower optimal dose (Boonyaratpalin S. et al. (1995) “Effects of peptidoglycan prepared from Brevibacterium lactofermentum on growth, survival, immune response, and tolerance to stress in black tiger shrimp, Penaeus monodon.” In: Disease in Asian Aquaculture II. M. Shariff, J. R. Arthur & R. P. Subasinghe (eds.), p. 469-477. Fish Health Section, Asian Fisheries Society, Manila, Philippines).

Surprisingly, we could show that a concentration of 0.5 g-0.01 g PG kg⁻¹ diet in combination with nucleotides was effective to significantly improve survival of infected fish in experiment 1 and 2. Moreover, peptidoglycan was effective in a concentration as low as 0.05 g kg⁻¹ feed in improvement of post-challenge survival in experiment 2 without an addition of nucleotides.

Oral administration of peptidoglycan in combination with nucleotides during the pre-challenge period in experiment 2 gave significant protection in survival of fish at the end of the post-challenge period of up to 78% RPS (survival probability p<0.001, Log Rank-Wilcoxon).

Best results were achieved when peptidoglycan was administered to the fish in a low concentration (0.001% PG; final dose 6 mg kg⁻¹ body weight) in combination with nucleotides during the whole pre-challenge phase.

The results of the experiments suggest that adequate levels of nucleotides administered along with the immune stimulant are probably beneficial for the fish in periods of challenges or stress by infections. These effects may be due to nucleotides becoming limiting when cell populations are rapidly increased e.g. during periods of immune system stimulation.

Negative effects on immunity due to over-exposure to peptidoglycan have been reported in the literature (Matsuo & Miyazono, (1993) reported that oral administration of PG at 0.2 and 2 mg/kg diet enhances the disease resistance of rainbow trout against V. anguillarum infection but a longer administration than 28 days can lead to decrease in disease resistance.

The results of the experiments suggest that nucleotides may compensate for the these negative effects related to an over-exposure and/or long-term by peptidoglycan.

The present invention thus represents an important improvement for the health management of fish.

It will be appreciated that the features of the invention described in the foregoing can be modified without departing from the scope of the invention.

### DEFINITIONS OF TERMS

The term peptidoglycan comprises all commonly described and not yet described compounds belonging to the substance group of peptidoglycans.

Nucleotides comprise any known phosphor ester of a nucleoside such as AMP, GMP, UMP, CMP, UMP.

Conventional feed ingredients are feed ingredients which are commonly used in feed compositions for a specific animal species such as lipids, proteins, vitamins, carbohydrates, minerals, etc.

A functional feed which can be similar in appearance to, or may be, a conventional food that is consumed as part of a usual diet, and is demonstrated to have physiological benefits and/or reduce the risk of certain diseases beyond basic nutritional functions, i.e. by comprising bioactive compounds such as nucleotides and peptidoglycan.

Salmonids are fish belonging to the family of Salmonidae. Representative examples are Atlantic salmon (Salmo salar), Rainbow trout (Onchorynchus mykiss), Coho salmon (Onchorynchus kisutch).

The term infectious disease includes commonly known infectious diseases of animals e.g. caused by bacteria such as Piscirikettsia salmonis, Moritella viscosa, Francisella sp, Mouth Rot, Streptococcal infections, Vibrio infections; Pancreas disease, NSAV, gill inflammation (GI), heart and skeletal muscle inflammation (HSMI), infectious salmonid anaemia (ISA) virus, Saprolegniosis, and sealice infestation.

The term recovery means that the health of an animal is restored after it has been affected by a disease.

1. A feed composition comprising peptidoglycan, nucleotides, and conventional feed ingredients; wherein said peptidoglycan is in the range of 0.001-0.01%

2. The feed composition according to claim 1, wherein said peptidoglycan is in the range of 0.001-0.005% and most preferably 0.005% by weight.

3. The feed composition according to claim 1, wherein the nucleotides are in the range of 0.05%-1% by weight, preferably 0.1-0.5% by weight and most preferably of 0.2% by weight.

4. The feed composition according to claim 1, wherein the weight ratio of nucleotides to peptidoglycan is more than 4, more preferable more than 20, and most preferable more than 40.

5. (canceled)

6. The feed composition according to claim 1, wherein the weight ratio of nucleotides to peptidoglycan is more than 20, and the amount of peptidoglycan is in the range of 0.001-0.01% of the weight of the feed composition.

7. (canceled)

8. (canceled)

9. (canceled)

10. The feed composition according to claim 1, wherein the amount of nucleotides is about 0.2% and the amount of peptidoglycan is about 0.005% of the total weight of the feed composition.

11. The feed composition according to claim 1 wherein, the nucleotides are selected from the group consisting of adenosine monophosphate, cytidine monophosphate, guanosine monophosphate, uridine monophosphate, and thymidine monophosphate.

12. The feed composition according to claim 1 further comprising immune stimulating and/or anti-inflammatory ingredients.

13. A method of prophylaxis or treatment of infectious diseases in animals comprising administering to an animal in need of such treatment, a pharmaceutically effective amount of a pharmaceutical or nutritional composition comprising peptidoglycan, nucleotides and conventional feed ingredients.

14. The method of claim 13, wherein said peptidoglycan is in the range of 0.001-0.01%, preferably 0.001-0.005% and most preferably 0.005% by weight.

15. The method of claim 13, wherein the nucleotides are in the range of 0.05%-1% by weight, preferably 0.1-0.5% by weight and most preferably of 0.2% by weight.

16. The method of claim 13, wherein the weight ratio of nucleotides to peptidoglycan is more than 4, more preferable more than 20, and more preferable more than 40.

17. The method of claim 13, wherein the weight ratio of nucleotides to peptidoglycan is more than 40.

18. The method of claim 13, wherein the weight ratio of nucleotides to peptidoglycan is more than 20, and the amount of peptidoglycan is in the range of 0.001-0.01% of the weight of the feed composition.

19. (canceled)

20. (canceled)

21. The method of claim 13, wherein the amount of nucleotides is about 0.2% and the amount of peptidoglycan is about 0.005% of the total weight of the feed composition.

22. The method of claim 13, wherein the nucleotides are selected from the group consisting of adenosine monophosphate, cytidine monophosphate, guanosine monophosphate, uridine monophosphate, thymidine monophosphate.

23. The method of claim 13, wherein the composition further comprises other immune stimulating and/or anti-inflammatory ingredients.

24. (canceled)

25. (canceled)

26. The method of claim 13 wherein, the infection is caused by bacteria such as Piscirikettsia salmonis, Moritella viscosa, Francisella sp, Mouth Rot, Streptococcal infections, Vibrio infections; Pancreas disease, NSAV, gill inflammation (GI), heart and skeletal muscle inflammation (HSMI), infectious salmonid anaemia (ISA) virus, Saprolegniosis, and sealice infestation.

27. The method of claim 13 wherein, the animal is an aquatic animal.

28. The method of claim 27, wherein the aquatic animal is a fish, crustacean or mollusk.

29. The method of claim 28, wherein the fish is a salmonid, flat fish, or any other culturable fish species.

30. (canceled)

31. (canceled)

32. (canceled)

33. The method of claim 13, for improved treatment of an infection in combination with antibiotics or prior to an antibiotic treatment.

34. A method for feeding of fish, which are susceptible to an infection by administering to a fish in need thereof a feed composition comprising peptidoglycan, nucleotides and other feed ingredients prior to challenge by an infection, during the infection or after the fish has been infected.

35. The method of claim 34 wherein, the composition is fed for a period of 1-12 weeks prior to the infection, more preferably of 2-6 weeks, most preferably of 4 weeks.

36. The method of claim 34 wherein the fish are fed a conventional feed composition further comprising nucleotides for a defined period followed by feeding of the composition according to claim 1.

37. (canceled)

38. (canceled)

39. The method of claim 34 wherein, the infection is caused by bacteria such as Piscirikettsia salmonis, Moritella viscosa, Francisella sp, Mouth Rot, Streptococcal infections, Vibrio infections; Pancreas disease, NSAV, gill inflammation (GI), heart and skeletal muscle inflammation (HSMI), infectious salmonid anaemia (ISA) virus, Saprolegniosis, and sealice infestation.
